(19) United States
(12) Patent Application Publication
(10) Pub. No.: US 2022/0063453 A1
(43) Pub. Date: Mar. 3, 2022

(54) Systems And Methods For Controlling Vehicle Seat Position

(71) Applicant: Ford Global Technologies, LLC, Dearborn, MI (US)

(72) Inventors: Juan Alejandro Hernandez, Mexico City (MX); Carlos Alberto Lopez, Tlalnepantla (MX)

(73) Assignee: Ford Global Technologies, LLC, Dearborn, MI (US)

(21) Appl. No.: 17/008,595

(22) Filed: Aug. 31, 2020

(51) Int. Cl. B60N 2/16; B60R 1/02; B60N 2/66; B60K 35/00; B60Q 9/00; G01G 19/44; G01G 19/52; A61B 5/00

(52) U.S. Cl. CPC B60N 2/16; B60R 1/02; B60N 2/66; B60K 35/00; B60K 2370/12; G01G 19/44; G01G 19/52; A61B 5/6893; B60K 2370/152; B60Q 9/00

(57) ABSTRACT

Systems and methods for controlling vehicle seat position are disclosed herein. An example device can be configured to determine seat configuration parameters of a seat of a vehicle, determine seat weight distribution of a user, automatically adjust the seat configuration parameters in such a way that the seat weight distribution changes as the user is operating the vehicle, and automatically adjust a mirror position of the mirror of the vehicle in response to the automatic adjustment of the seat configuration parameters.

### BACKGROUND

Sitting in a car for an extended time can lead to physical discomfort. Even without medical issues, a user can experience discomfort and soreness from sitting in a seat of a car for an extended period. In some instances, a seat shape or configuration may not adequately correspond to a body shape of a user leading to discomfort. When discomfort begins, people may try to move their bodies across the seating area, for example, by stretching their legs, straightening their backs, or shifting their weight. While these actions can help to distribute the body weight differently and cause changes in muscle responses, these body position changes may not be held for long periods of time because the seat position may provide inadequate support. Thus, attenuation of discomfort may last for a short period and then the user may need to let their body return to the position determined by the seat.

### BRIEF DESCRIPTION OF THE DRAWINGS

The detailed description is set forth regarding the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an illustrative architecture in which techniques and structures for providing the systems and methods disclosed herein may be implemented.

FIG. 2 illustrates a graphical user interface for toggling a seat position feature on and off, as well as a selection for entering anthropometric data.

FIG. 3 illustrates another graphical user interface for toggling a seat position feature on and off

FIG. 4 is a flowchart of a method of the present disclosure.

FIG. 5 is a flowchart of another method of the present disclosure.

### DETAILED DESCRIPTION

### Overview

The present disclosure is generally directed to systems and methods that automatically adjust seat configuration parameters in response to seat weight distribution created by a user sitting in the seat. The seat weight distribution can be sensed, and periodic automatic adjustment to seat configuration parameters can be executed to vary seat position and reduce physical discomfort. Stated otherwise, adjustment of seat configuration parameters can change the seat weight distribution to reduce discomfort caused when the user sits in the seat for long periods during vehicle operation. Seat weight distribution may be determined from pressure/weight sensors embedded into various locations of a seat, such as a bucket or lumbar region of the seat.

The adjustments to the seat configuration parameters can be based on anthropometric data or medical data of the user. For example, the anthropometric data can include physical dimensions of the user. Medical information can include data related to back injuries, thrombosis risk factors, age, physical activity level, body weight, and so forth.

When an adjustment to the seat configuration parameters occurs, it may cause a misalignment between the user and a mirror of the vehicle, such as an external side mirror or rearview mirror. Thus, adjustments of seat configuration parameters can result in automatic adjustments to a mirror position.

### Illustrative Embodiments

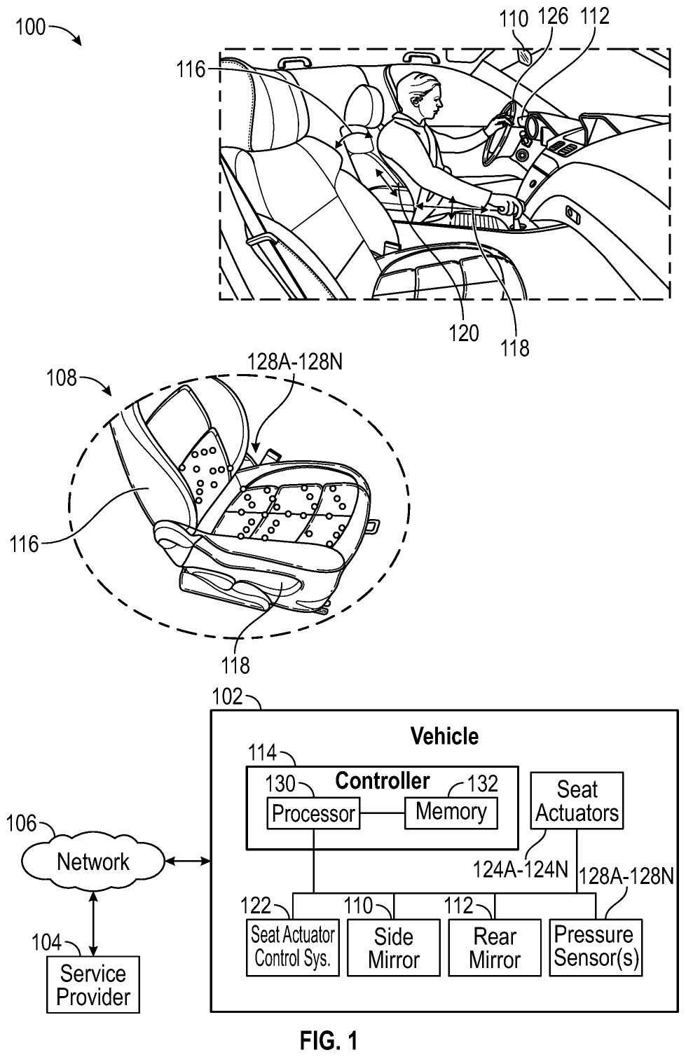

Turning now to the drawings, FIG. 1 depicts an illustrative architecture 100 in which techniques and structures of the present disclosure may be implemented. The architecture 100 can include a vehicle 102, a service provider 104, and a network 106. The vehicle may be any vehicle for any purpose, including personal, construction, commercial, delivery, etc., whether an internal combustion engine, electric vehicle, or hybrid. The network 106 may include any one or a combination of multiple different types of networks, such as cable networks, the Internet, cellular networks, wireless networks, and other private and/or public networks. In some instances, the network 106 may include cellular, Wi-Fi, or Wi-Fi direct. In other embodiments, components of the environment can communicate using short-range wireless protocols such as Bluetooth, near-field, infrared, and the like.

The vehicle 102 can generally include a seat 108, a side view mirror 110, a rearview mirror 112, and a controller 114. The seat 108 can comprise a back section 116, a bucket section 118, a lumbar support 120, and a seat actuator control system 122 that is configured to control a plurality of seat actuators 124A-124N. The seat actuator control system 122 can be used to control one or more of the seat components of the seat. A seat actuator can include an electronically controlled mechanism that can be used to selectively adjust a position of a seat component. For example, a first seat actuator can be used to control inclination or angle of the back section 116 of the seat 108. Another one of the seat actuators can be used to selectively adjust a lumbar support of the seat 108 (could include positioning of the lumbar support or inflation/deflation). Another seat actuator can be used to adjust proximity (e.g., distance) between the seat 108 and the steering wheel 126. The descriptions of seat actuators disclosed herein are not intended to be limiting in any way. Some seats may include additional or fewer movable components associated with seat actuators. For example, some seats may include lateral support members that can extend and retract.

Regardless of the configuration, these electronically controlled mechanisms can be controlled through automatic adjustment of seat configuration parameters by the controller 114. In general, seat configuration parameters may include settings for a seat actuator that determine a position or other operating parameter for a seat component. For example, a value of eighteen inches would be a seat configuration parameter that indicates how far the seat 108 and the steering wheel 126 should be spaced apart. This value could be expressed as a position value for the seat 108 as it moves along a track or other similar guide mechanism. The seat actuator control system 122 can use the seat configuration parameters to adjust movement/position of any of the plurality of seat actuators 124A-124N.

Pressure sensors 128A-128N can be embedded into various areas of the seat 108. The pressure sensors can be referred to generally as weight sensors. Pressure sensors 128A-128N can monitor a strength distribution, particularly in the lumbar and the down zones of the seat 108. The pressure sensors 128A-128N can be distributed across these regions in a matrixial configuration and can include strength gage sensors.

For example, a first portion of the pressure sensors 128A-128N can be embedded into the bucket section 118, while a second portion of the pressure sensors 128A-128N can be embedded into the lumbar support 120 area of the back section 116. While pressure sensors have been described, other sensors capable of sensing the weight of a user can likewise be utilized. Generally, the output of the pressure sensors 128A-128N can be used to create a current pressure profile for the seat. This pressure profile may be referred to as a seat weight distribution for the user.

As noted above, change in seat configuration may result in changes in mirror position. For example, the side-view mirror 110 and the rearview mirror 112 may each be electronically controllable. As will be discussed below, a position or orientation of the side view mirror 110 and/or the rearview mirror 112 can be selectively adjusted as the seat configuration parameters are varied, which results in movement of seat component(s) such as the back section 116, the bucket section 118, and/or the lumbar support 120. The movement of the mirrors can be based on the anthropometric data of the user in some instances.

Each of the side view mirror 110 and the rearview mirror 112 can be controlled individually through electronically controlled mechanisms such as motors or servos. In some instances, these mirrors can be controlled through switches made available to the user. The mirrors can also be actuated through the controller 114 as will be discussed in detail below.

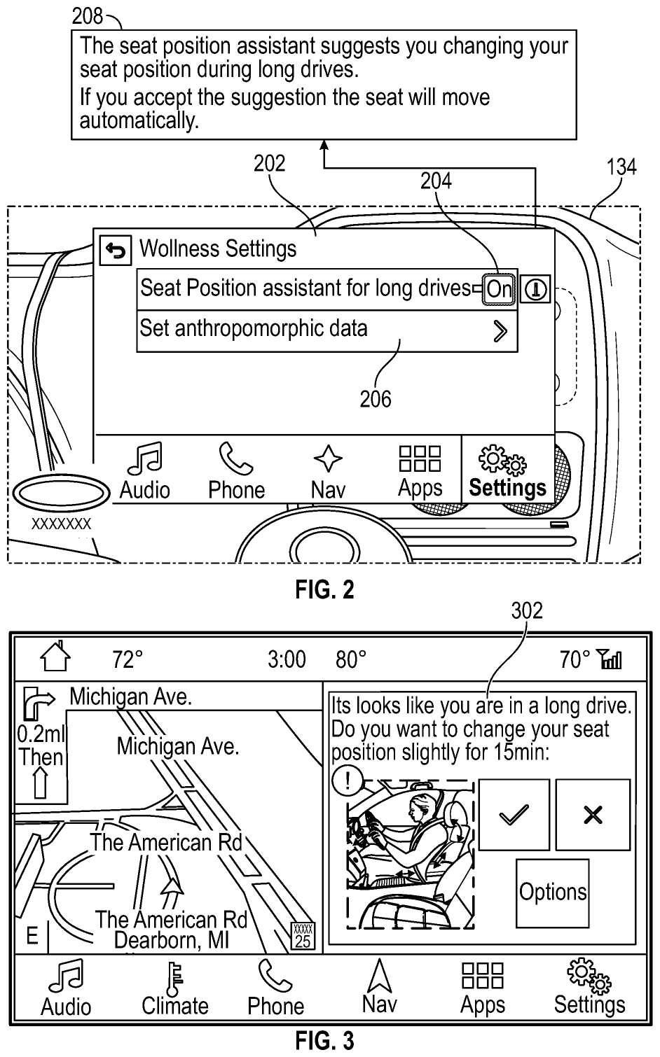

The service provider 104 can be used to provide user records for storing information such as anthropometric data and health or medical data of the user. Generally, the anthropometric data can be obtained from user input as will be discussed in greater detail below with reference to FIG. 2. Medical data can be obtained from the user directly or from a medical service provider. In some instances, the anthropometric data and/or health or medical data can be stored locally at the vehicle level.

Generally, the controller 114 can comprise a processor 130 and memory 132. The memory 132 stores instructions that can be executed by the processor 130 to perform any of the seat adjustment methods disclosed herein. The controller 114 can be configured to communicate over the network 106 with any of the components of the architecture such as the service provider 104. The controller 114 can incorporate or use any vehicle communication device to transmit and/or receive data over the network 106. When referencing operations performed by the controller, it will be understood that this includes the execution of instructions stored in the memory 132 by the processor 130.

Some embodiments disclose the controller as controlling or causing various components of the vehicle to be controlled. It will be understood that this can include direct control of a component by the controller or indirect control of the component through another vehicle controller, device, system, or assembly. In one non-limiting example, the controller could directly control a human-machine interface (HMI) 134 of the vehicle to display graphical user interfaces (GUIs). This could include the controller generating and transmitting the GUI for display on the HMI, or causing a dedicated processor of the HMI to generate and display the GUI. Again, this example is not intended to be limiting. In another example, the controller 114 can cause movement of seat components indirectly through communication with the seat actuator control system 122.

Also, it will be understood that while methods disclosed herein as being executed at the vehicle level can also be executed at a service provider level. In some instances, methods can be cooperatively executed at both the vehicle level and the service provider level in combination.

FIG. 2 illustrates an example GUI 202 that can be displayed on the HMI 134 of the vehicle 102. The GUI 202 can include a selectable unit 204 that allows a user to toggle a seat position assistant feature on and off. The GUI 202 can include a selectable unit 206 that allows a user to enter their anthropometric data and/or medical data for storage and use. A notification 208 can be presented to the user that informs them that their seat position may change periodically when the position assistant feature is on.

Initially, when a user sits in the seat 108, a set of current seat configuration parameters for the seat 108 can be obtained. The output from the pressure sensors 128A-128N can also be obtained. The controller 114 can utilize these data to determine how long the user has been sitting in their current position in the seat 108. If the user has been sitting for a threshold period of time, such as thirty minutes without adjusting their position, the controller 114 can suggest a position change to the user. The threshold time frames disclosed herein are for descriptive purposes and are not intended to be limiting. As illustrated in FIG. 3, the controller 114 can cause the display of a GUI 302 that queries the user if they desire to turn on the position assistant feature. The prompt to turn on the position assistant feature can be triggered when the controller 114 determines that the user has been sitting in the same (or substantially the same) position for a threshold time. The prompt to turn on the position assistant feature can be triggered when the controller 114 determines that the user is on a long drive. This data can be determined from a vehicle navigation system or a connected mobile device with a mapping feature. The length of drive that can initiate the prompt can be set at any desired distance or estimated time of arrival.

Referring back to FIG. 1, in an example use case, the controller 114 can be configured to activate the position assistant feature. As noted above, this can occur based on user selection or can be triggered automatically based on trip length. In another example, the controller can collect output from the pressure sensors 128A-128N and use this output to determine if the user has become uncomfortable. For example, when a user is uncomfortable, they may begin to shift their weight in the seat 108 or perform other maneuvers such as leg stretching or back straightening. Each of these movements can be identified from output of the pressure sensors 128A-128N. The position assistant feature can be triggered when the controller 114 detects that the user is uncomfortable. In some instances, the controller 114 can utilize machine learning and training to determine when the user is likely to become uncomfortable. Rather than automatically using the position assistant feature, the controller 114 can query the user regarding seat adjusting feature use as illustrated in FIGS. 2 and 3.

Once the position assistant feature has been activated, the controller 114 can automatically adjust the seat configuration parameters in such a way that the seat weight distribution changes as the user is operating the vehicle. For example, the lumbar support 120 can be adjusted to increase or decrease support, or the position of the lumbar support 120 could be moved upwardly or downwardly. A pitch of the bucket section 118 can be altered. A position of the bucket section 118 can be altered to be closer or further away from the steering wheel 126. An angle of the back section 116 can be altered. In some instances, each of the seat components can be moved or repositioned at least to some degree. In other instances, only a portion of the seat components are moved or repositioned at least to some degree. The controller 114 can determine which of the seat components require movement or repositioning based on the output of the pressure sensors 128A-128N. For example, as the seat components are moved or repositioned, the output of the pressure sensors 128A-128N can be measured by the controller 114. When the output of the pressure sensors 128A-128N indicates that the current pressure profile for the seat has changed, the position of the seat 108 can be maintained for a period of time. For example, the controller 114 can cause the seat 108 to be reconfigured according to a first set of seat configuration parameters to a second set of seat configuration parameters for a period of fifteen minutes. Again, this is merely an example time frame. After expiration of the time frame, the controller 114 can cause the seat 108 to be reconfigured according to the second set of seat configuration parameters back to the first set of seat configuration parameters. In some instances, the seat configuration parameters can be based on the user's anthropometric data or medical information. For example, the controller 114 can increase the firmness and the position lumbar support 120 based on a noted back injury of the user.

In addition to seat component positioning, the controller 114 can also activate other seat features such as seat heating, seat cooling/ventilation, and/or seat massaging features, if present. The anthropometric data can be used to place limits on seat movement. For example, a height of the user can be used to limit the displacement of the seat. If the user is short, the seat may not be moved more than a predetermined distance away from the steering wheel 126, as an example.

When seat configuration parameters are adjusted, the seat actuator control system 122 can implement the seat configuration parameters by causing the plurality of seat actuators 124A-124N to move seat components in response. During the movement of seat components, the controller 114 can cause movement of the mirrors, such as the side view mirror 110 and/or the rearview mirror 112. As noted above, the controller 114 can utilize machine learning and training to associate changes in seat configuration parameters to mirror position(s). For example, moving the seat 108 backwardly could result in panning of the side view mirror 110 horizontally. Moving the seat upwardly or downwardly could result in panning of the side view mirror 110 vertically. Similarly, changes in the position of the rearview mirror 112 can be made as well, such as when a vertical or horizontal position of the seat 108 changes. In sum, the controller 114 can automatically adjust a mirror position of a mirror of the vehicle correspondingly in response to the automatic adjustment of the seat configuration parameters.

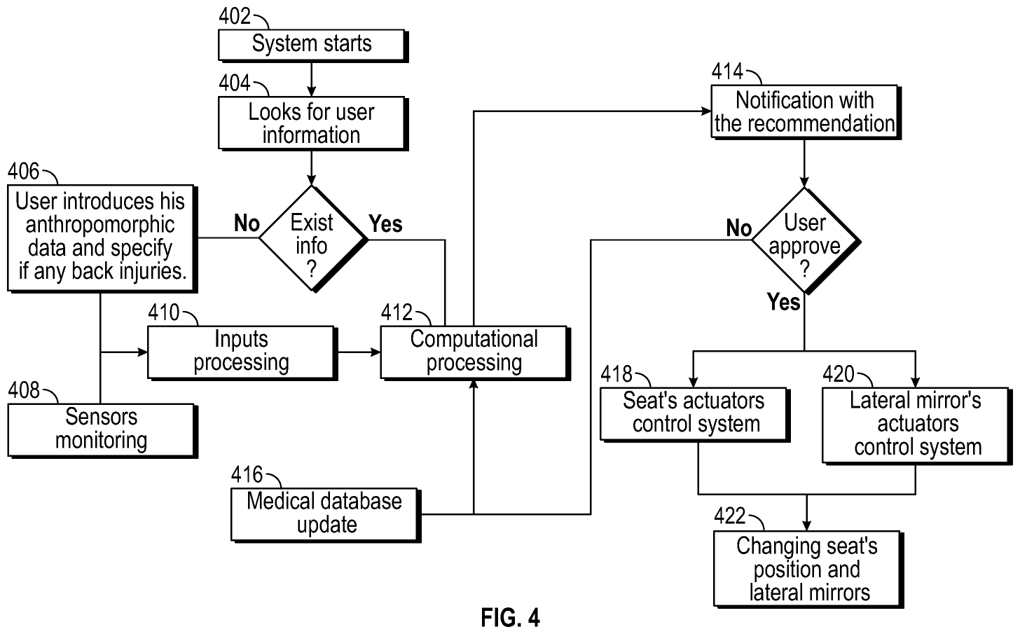

FIG. 4 is a flowchart of an example method of the present disclosure. The method can initiate in step 402 with activation of the vehicle and initiation of the position assistant feature using any of the methods disclosed herein. When the system starts the user can create a user profile. When the user starts to drive they can select their profile previously created.

The method can include a step 404 of determining if anthropometric and/or medical data exist for the user. If not, the user can be prompted to enter their anthropometric and/or medical data in step 406 using an HMI or other equivalent data input mechanism of the vehicle. In this step, the user can create a new profile and he/she can introduce anthropomorphic data and also can specify if the user has back problems or other medical issues. The sensors associated with the seat of the vehicle can be monitored in step 408.

Inputs (e.g., outputs of the pressure sensors) are processed in step 410, along with computational processing in step 412 where seat position data and pressure sensor data are processed. The user can be notified with a suggestion to move the seat position in step 414. When the user does not agree to have automatic seat adjustments made, any collected data can be fed back into a medical database (at the service provider or locally in the vehicle) for updating of medical data for the user in step 416.

The medical data can be updated to improve the parameters used to avoid new kinds of injuries, and can also be used to generate statistics about driver habits. This medical database contains information about what kind of driver positions can generate injuries and how much time the body tolerates a seat configuration without injury or irritation. The medical database can include information about recommended seat positions for a user with back problems depending on the type of injuries.

Inputs processed in this method can relate to the anthropomorphic user's data. In some instances, the system can pre-catalog the parameters of the user according to important characteristics like age, complexion, and injuries, for example. Computational processing can determine operations such as assigning a recommended seat position for the user, depending on their anthropometric characteristics and the status of the pressure/stress sensors. The input information can be processed as a regulated variable that can be linked with the medical database. The medical database can be used as an adaptable control scheme that can be improved constantly and also can be adapted for the user and their medical needs.

When the user does not agree to have automatic seat adjustments made, the seat actuator control system can make adjustments in seat components based on changes to the seat configuration parameters in step 418. A mirror actuator system can make adjustments to mirror position(s) to correspond with the seat component adjustments in step 420, which result in the changes to both the seat components and the mirror(s) in step 422.

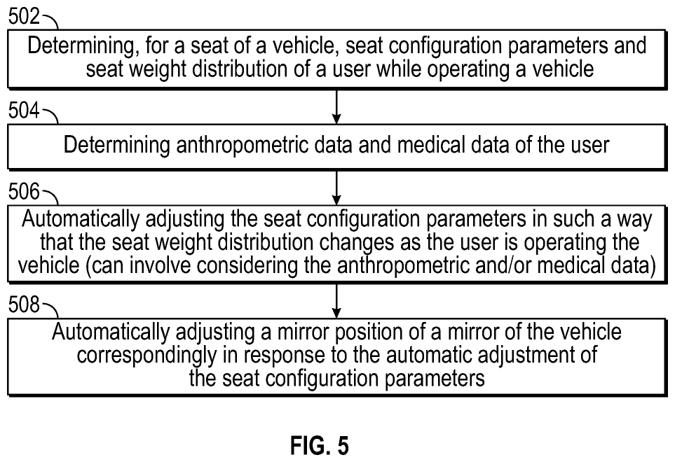

FIG. 5 is a flowchart of another example method. The method can include a step 502 of determining, for a seat of a vehicle, seat configuration parameters and seat weight distribution of a user while operating a vehicle. The method can include a step 504 of determining anthropometric data and medical data of the user.

Next, the method can include a step 506 of automatically adjusting the seat configuration parameters in such a way that the seat weight distribution changes as the user is operating the vehicle. In some instances, the automatic adjustment to the seat configuration parameters is based on the anthropometric data and the medical data of the user. In some instances, the seat weight distribution change can be set according to anthropometric data or medical data for the user in some instances. The change in seat configuration parameters may reduce fatigue or pain that might occur if the user were to sit in the same configuration for a long period of time. The seat configuration parameters can include any of seat inclination, lumbar, seat proximity, seat height, and combinations thereof.

The method can include a step 508 of automatically adjusting a mirror position of a mirror of the vehicle correspondingly in response to the automatic adjustment of the seat configuration parameters. That is, when the seat configuration parameters cause the seat to move, corresponding corrective changes in mirror positioning may occur.

The method can include displaying a graphical user interface on a human machine interface of the vehicle that allows a user to select automatic adjustment of the seat configuration parameters and the mirror position. A user can be allowed to enter anthropometric or medical data into the human machine interface. The graphical user interface can be displayed according to a schedule. For example, the GUI can be displayed every thirty minutes to encourage the user to change their seat configurations. In some instances, an audible or visual notification can be output to the user that reminds the user that the seat configuration parameters are adjustable.

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiment.

Computer-executable instructions comprise, for example, instructions and data which, when executed at a processor, cause a general purpose computer, special purpose computer, or special purpose processing device to perform a certain function or group of functions. The computer-executable instructions may be, for example, binaries, intermediate format instructions such as assembly language, or even source code. A “network” is defined as one or more data links that enable the transport of electronic data between computer systems and/or modules and/or other electronic devices. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the present disclosure may be practiced in network computing environments with many types of computer system configurations, including in-dash vehicle computers, personal computers, desktop computers, laptop computers, message processors, handheld devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by any combination of hardwired and wireless data links) through a network, both perform tasks. In a distributed system environment, program modules may be located in both the local and remote memory storage devices.

It should be noted that the sensor embodiments discussed above may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

What is claimed is:

1. A method, comprising:
determining, for a seat of a vehicle, seat configuration parameters and seat weight distribution of a user while operating a vehicle;
automatically adjusting the seat configuration parameters to change the seat weight distribution as the user is operating the vehicle; and
automatically adjusting a mirror position of a mirror of the vehicle correspondingly in response to the automatic adjustment of the seat configuration parameters.

2. The method according to claim 1, further comprising determining anthropometric data and medical data of the user, wherein the automatic adjustment to the seat configuration parameters is based on the anthropometric data and the medical data of the user.

3. The method according to claim 1, wherein the seat configuration parameters include seat inclination, lumbar, seat proximity, seat height, and combinations thereof.

4. The method according to claim 1, further comprising displaying a graphical user interface on a human machine interface of the vehicle that allows the user to select automatic adjustment of the seat configuration parameters and the mirror position.

5. The method according to claim 4, further comprising allowing the user to enter anthropometric data into the human machine interface.

6. The method according to claim 4, wherein the graphical user interface is displayed according to a schedule.

7. The method according to claim 1, further comprising outputting an audible or visual notification to the user that reminds the user that the seat configuration parameters are adjustable.

8. A system, comprising:
a seat of a vehicle, wherein the seat comprises weight sensors and seat actuators;
a mirror associated with the vehicle; and
a controller comprising a processor and a memory, wherein the memory stores instructions that are executed by the processor to:
determine seat configuration parameters related to the seat actuators;
determine seat weight distribution of a user using output of the weight sensors;
automatically adjust the seat configuration parameters to cause a corresponding change in the seat actuators to change the seat weight distribution of the user; and
automatically adjust a mirror position of the mirror of the vehicle in response to the automatic adjustment of the seat configuration parameters.

9. The system according to claim 8, wherein the mirror is a side mirror of the vehicle.

10. The system according to claim 8, wherein the processor is configured to automatically adjust a mirror position of a rearview mirror.

11. The system according to claim 8, wherein the processor is configured to determine anthropometric data and medical data of the user, wherein the automatic adjustment to the seat configuration parameters is based on the anthropometric data and the medical data of the user.

12. The system according to claim 8, wherein the seat configuration parameters include seat inclination, lumbar, seat proximity, seat height, and combinations thereof.

13. The system according to claim 8, wherein the weight sensors are distributed across a bucket portion and a lumbar region of the seat.

14. The system according to claim 11, wherein the processor is configured to obtain the medical data of the user from a database.

15. The system according to claim 8, wherein the processor is configured to cause a command system of the vehicle to output an audible or visual notification to the user that reminds the user that the seat configuration parameters are adjustable.

16. A device, comprising:
a processor; and
a memory configured to store instructions that are executed by the processor to:
determine seat configuration parameters of a seat of a vehicle;
determine seat weight distribution of a user;
automatically adjust the seat configuration parameters to change the seat weight distribution as the user is operating the vehicle; and
automatically adjust a mirror position of a mirror of the vehicle in response to the automatic adjustment of the seat configuration parameters.

17. The device according to claim 16, wherein the processor is configured to determine anthropometric data and medical data of the user, wherein the automatic adjustment to the seat configuration parameters is based on the anthropometric data and the medical data of the user, and wherein the seat configuration parameters include any one or more of seat inclination, lumbar, seat proximity, and seat height.

18. The device according to claim 17, wherein the processor is configured to display a graphical user interface on a human machine interface of the vehicle that allows the user to select automatic adjustment of the seat configuration parameters and the mirror position.

19. The device according to claim 18, wherein the processor is configured to allow the user to enter anthropometric data into the human machine interface when the anthropometric data is not present in a medical database that is accessible to the processor.

20. The device according to claim 19, wherein the graphical user interface is displayed according to a schedule.
